(19) United States
(12) Patent Application Publication
(10) Pub. No.: US 2023/0329582 A1
(43) Pub. Date: Oct. 19, 2023

(54) PATIENT MONITORING SENSOR PAD

(71) Applicant: Rondish Company Limited, Kwai Chung, N.T. (HK)

(72) Inventor: Steven Alfred Williams, Kwai Chung, N.T. (HK)

(21) Appl. No.: 18/005,032

(22) PCT Filed: Jul. 8, 2021
(86) PCT No.: PCT/IB21/56166

§ 371 Date: Jan. 10, 2023

Related U.S. Application Data
(60) Provisional application No. 63/050,607, filed on Jul. 10, 2020.

(51) Int. Cl. A61B 5/11; A61B 5/00

(52) U.S. Cl. CPC A61B 5/1115; A61B 5/6892

(57) ABSTRACT

A sensor pad is adapted to be positioned atop or under a mattress of a patient's bed or chair, as part of a monitoring system that provides a signal to a caregiver when the patient rises from the bed, which can be used for multiple patients. The under-mattress sensor pads can be used for a long period of time as the under-mattress sensor pads includes a relatively stiff material. The under-mattress sensor pads can functionally couple to corded and wireless monitoring systems regardless of manufacturer. The under-mattress sensor pads can also be adapted to provide multiple alarm functions, which may be selectable by user operation.

### CROSS-REFERENCE TO RELATED APPLICATION

This application claims priority to U.S. Provisional Patent Application No. 63/050,607, filed Jul. 10, 2020, entitled “PATIENT MONITORING SENSOR PAD”, the content of which is hereby incorporated by reference herein in its entirety.

### BACKGROUND

The present disclosure relates to a sensor pad, in particular, a pad for detecting when an individual exits a chair or a bed.

A serious problem encountered by operators of hospitals, nursing homes, retirement centers, and other facilities that care for patients with limited mobility is that patients who leave their beds without assistance may subsequently trip or fall. Patients that are heavily medicated or sedated are particularly susceptible to falls. For these reasons, much consideration has been given to systems for monitoring patients and providing a warning that a patient is rising from or has left his or her bed.

One type of currently used sensor pad is one that is positionable on a mattress of a bed or seat of a chair. The sensor pad is communicatively coupled to a monitoring device, such as a nurse call system. When pressure on the sensor pad is removed, such as when the patient removes himself or herself from the bed or chair, the sensor pad sends a notification to the monitoring device which provides a notification to the caregiver that the patient is no longer in bed or seated in the chair.

Currently, most of the sensor pads used in patient beds are placed above the mattress in a patient bed. Because of this positioning, current patient monitoring sensor pads are made of soft materials, that are comfortable to sit or lay on, and are more frequently exposed to fluids, friction, and other situations that may reduce the longevity of the monitoring sensor pads. Described below are patient monitoring sensor pads that are robust and may be placed under the mattress of a patient's bed.

Additional challenges facing users of patient monitoring sensor pads include compatibility and connection of the pad with various monitoring systems, as well as control of the alarm features of the pad. For example, bed pads from a particular manufacturer may not include electronic connections that are compatible with monitoring systems from another manufacturer, or they may not be compatible with wireless monitoring systems. Described below are patient monitoring sensor pads that have multiple alarm features, and are universally compatible with corded and wireless monitoring systems, regardless of the manufacturer or mode of connection (e.g. cord-to-cord, or cord-to-wireless transceiver). Also described are patient monitoring sensor pads that include an integrated monitoring and alarm system and thus do not require connection to an external monitoring system.

### SUMMARY

Disclosed is a patient monitoring pad, or patient monitoring sensor pad, that can be placed underneath the mattress in a patient's bed. The patient monitoring sensor pad can be used for multiple patients and over an extended period of time before degradation in the performance of the monitoring pad.

In one aspect, the patient monitoring sensor pad comprises a flexible first plate with conductive material printed thereon; a flexible second plate with conductive material printed thereon, wherein the first flexible plate and second flexible plate comprise zones of conductivity; wherein the zone of conductivity of the first plate is configured to make physical and electrical contact with the zone of conductivity of the second plate when a patient sits or lies on the patient monitoring sensor pad; a foam layer, wherein the foam layer is an electrically conductive foam and wherein an electrical resistance of the electrically-conductive foam varies as a function of an amount of force applied to the electrically conductive foam; a bottom layer below the second plate, the bottom layer being more rigid than the first and second; and a neck portion extending from the bottom layer, the neck portion including an internal cavity, wherein the internal cavity includes at least one electrical contact and an aperture, the aperture configured to be opened and closed via a fastening member. In some embodiments, the electrical contact is a control chip. In some embodiments, the internal cavity also includes a battery. In some embodiments, the control chip includes a monitoring system and alarm feature.

In another, interrelated aspect, a method of using a patient monitoring sensor pad is provided. The method includes positioning the bed pad atop a bed frame, and activating a monitoring system. In some embodiments, the monitoring system is included in the patient monitoring sensor pad. In some embodiments, the monitoring system is external to the monitoring pad.

Other features and advantages should be apparent from the following description of various embodiments, which illustrate, by way of example, the principles of the disclosure.

### BRIEF DESCRIPTION OF THE DRAWINGS

FIG. 1A is a schematic of a conventional mattress patient monitoring sensor pad, showing the materials in the pad and how they are arranged.

FIG. 1B shows the patient monitoring sensor pad of FIG. 1A positioned atop a bed mattress.

FIG. 2A is a schematic of a first embodiment of an under-mattress patient monitoring sensor pad, showing the materials in the pad and how they are arranged.

FIG. 2B shows the patient monitoring sensor pad of FIG. 2A positioned atop a bed frame atop which a mattress is to be positioned.

FIG. 3 is a schematic of a second embodiment of an under-mattress patient monitoring sensor pad, showing the layers or materials in the pad and how they are arranged.

FIG. 4 is a schematic of a third embodiment of an under-mattress patient monitoring sensor pad, showing the materials in the pad and how they are arranged.

FIG. 5A is a schematic of an exemplary pattern of conducting material printed on a first plate that could be part of an over-mattress patient monitoring sensor pad.

FIG. 5B is a schematic of an exemplary pattern of conducting material printed on a second plate that could be part of an over-mattress patient monitoring sensor pad.

FIG. 6A is a schematic of an exemplary pattern of movement detection zones of a first plate that could be part of an over-mattress patient monitoring sensor pad.

FIG. 6B is a schematic of an exemplary pattern of movement detection zones of a second plate that could be part of an over-mattress patient monitoring sensor pad.

FIG. 7 is a schematic of an exemplary electrical circuit that could be part of an on-board monitoring system of a patient monitoring sensor pad.

FIG. 8A is a schematic of an exemplary neck portion and electrical contact that could be part of a patient monitoring sensor pad.

FIG. 8B is a schematic of the neck portion and electrical contact of FIG. 8A connected to a wire for use in a monitoring system.

FIG. 8C is a schematic of the neck portion and electrical contact of FIG. 8A connected to a wireless transceiver of a monitoring system.

FIG. 9 is a schematic of a fourth embodiment of an under-mattress patient monitoring sensor pad, showing the materials in the pad and how they are arranged.

FIG. 10 is a schematic of an exemplary low-density foam layer that could be part of an under-mattress patient monitoring sensor pad.

FIG. 11 is a schematic of an exemplary outer shell that could be part of an under-mattress patient monitoring sensor pad.

FIG. 12A is a schematic top view of an exemplary patient monitoring chair sensor pad.

FIG. 12B is a schematic top view of an exemplary patient monitoring bed sensor pad.

### DETAILED DESCRIPTION

A patient monitoring sensor pad that is configured for use either atop, within or under the mattress of a patient's bed or under the cushion of a patient's chair is described. The under-mattress patient monitoring sensor pad is made of robust materials and is configured for use with multiple patients, as well as for a long lifetime. The over-mattress patient monitoring sensor pad is compatible with a variety of external patient monitoring and alarm systems, or may include an on-board monitoring and alarm system. Also described are systems that include such patient monitoring sensor pads, as well as methods of using such patient monitoring sensor pads.

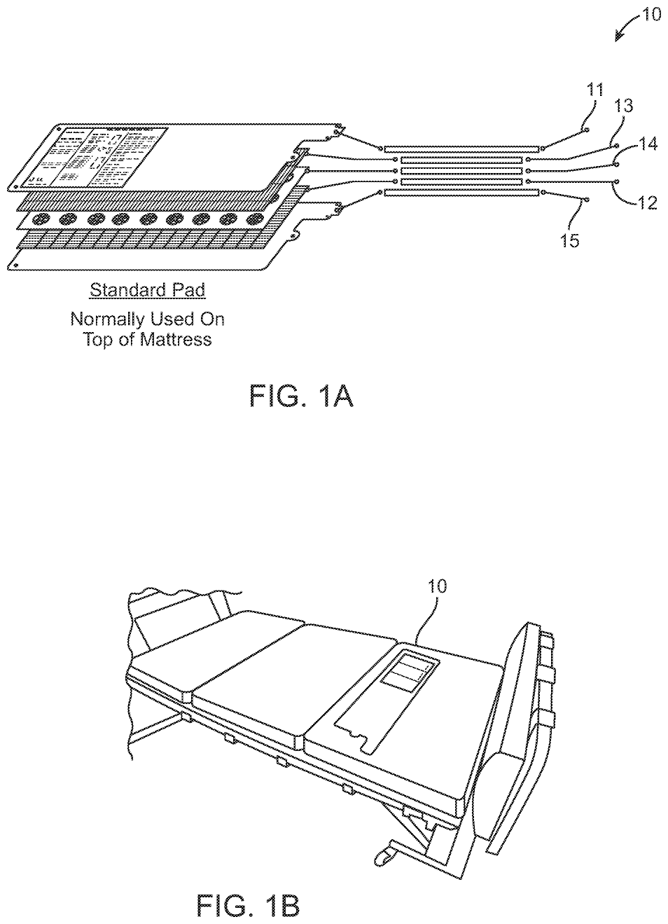

FIG. 1A shows a conventional patient monitoring sensor pad 10. FIG. 1B shows the conventional sensor pad 10 positioned atop a bed mattress. The conventional patient monitoring sensor pad 10 is configured for use on top of the mattress or cushion on which a patient lays or sits. Because the conventional patient monitoring sensor pad 10 is designed to be in close contact with a patient who lays atop the mattress, at times only separated by a sheet or thin layer of bedding, the conventional sensor pad 10 is made for patient comfort. What this means is that conventional patient monitoring sensor pads are made to be thin and soft. As a result, conventional patient monitoring sensor pads are flexible and bend easily. Also, the human body and variances in the shape and weight distribution of the body are relied upon for proper indications of the presence or absence of a patient.

In FIG. 1A, the conventional patient monitoring sensor pad 10 is shown as having five layers. The top 11 and bottom 15 layers can be for example Polyvinyl chloride (PVC), though any flexible, soft, durable, and water resistant material can be used. Between the top 11 and bottom 15 layers are two contact plates 13 and 12. The contact plates 13 and 12 can be hard thin plastic with a metallic or carbon coated layer.

A conventional patient monitoring sensor pad 10 is in reality not comfortable to lay on, and the conventional sensor pad can move as the patient moves, possibly moving the sensor pad into an undesirable position. Hygiene can also be a concern with conventional patient monitoring sensor pads because of the proximity to the patient and bodily fluids. Being on top of a patient's mattress or cushion can also expose the sensor pad to other conditions that could reduce the lifetime of the sensor pad. In some hospitals, conventional patient monitoring sensor pads are changed when a new patient occupies a bed because of the hygienic concerns.

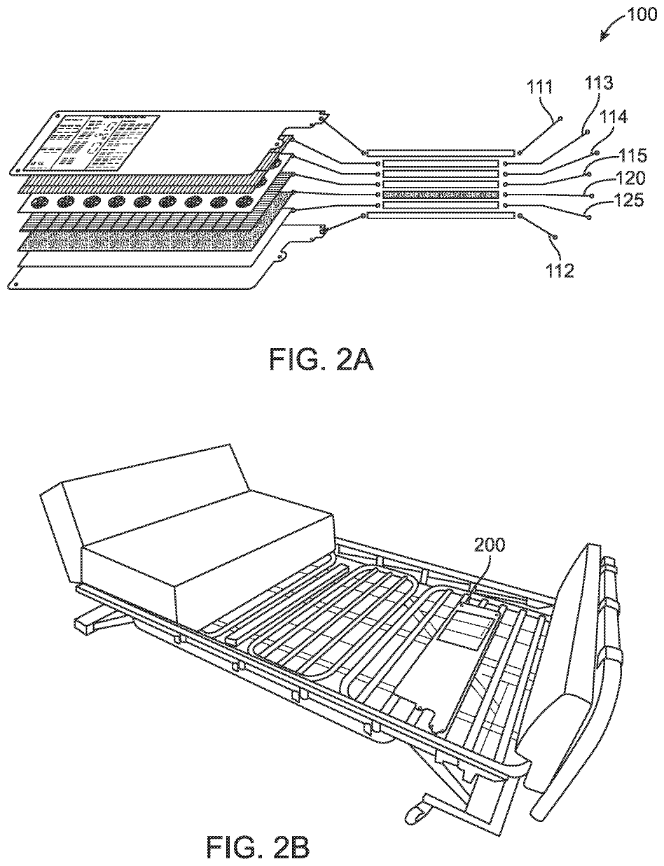

FIG. 2A shows a first exemplary embodiment of an under-mattress patient monitoring sensor pad 100. FIG. 2B shows the under-mattress patient monitoring sensor pad 100 positioned atop a bed frame atop which a mattress would be positioned. The bed frame has a plurality of slats or struts that extend across one side rail of the bed frame to the other, opposite side rail of the bed frame. The slats are thus positioned to extend orthogonal (i.e., at a right angle) relative to a long axis of the bed frame. This leaves orthogonal, elongated slots or openings between the slats. The side rails extend parallel to the long axis of the bed frame. As mentioned, in use a bed mattress is positioned atop the bed frame such that the patient monitoring sensor pad 100 is positioned between the slats of the bed frame and the mattress.

The configuration of the under-mattress patient monitoring sensor pad 100 accommodates the environment beneath a mattress or cushion. The under-mattress patient monitoring sensor pad 100 has more layers than the conventional patient monitoring sensor pad 10. The outer most layers 111 and 112 of the under-mattress patient monitoring sensor pad 100 can be for example PVC, but these layers 111 and 112 can be made of any flexible, soft, durable, and preferably water-resistant material. Immediately above the bottom layer 112 are a hard bottom plastic layer 125 and a heavy and/or rigid sponge or plastic layer 120. The rigid materials used to make the bottom plastic layer 125 and the heavy/rigid sponge/plastic layer 120 are selected to prevent the slats or strutted frame of a patient bed from causing false alarms or a non-functioning state in the sensor pad. In an embodiment, at least one of the layers below the contact plates is more rigid than either the contact plate(s) or the sponge layer. In an embodiment, a bottom most layer of the pad is sufficiently rigid such that it will not deform when a patient of at least 200 pounds lies on the mattress above the pad. Below the top layer 111 and above the heavy/rigid sponge or plastic layer 120 are two contact plates 113 and 115. The contact plates 113 and 115 may be configured to contact one another when a force is applied to the pad, such as when a patient lays on the mattress. The contact plates may be made of a flexible material such that they can at least partially flex or deform and contact one another. An electrically conductive material is printed on the contact plates.

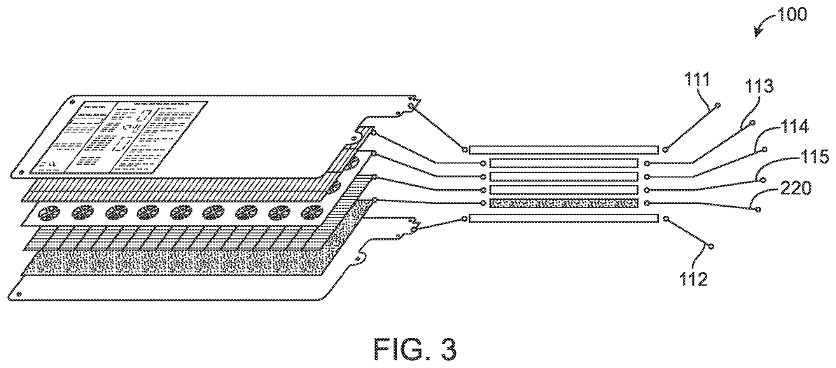

FIG. 3 shows a second exemplary embodiment of an under-mattress patient monitoring sensor pad 200. Like the under-mattress patient monitoring sensor pad 100 shown in FIG. 1, the under-mattress patient monitoring sensor pad 200 has outer most layers 211 and 212 that surround two contact plates 213 and 215. The two contact plates 213 and 215 may be configured to come in contact and close a circuit when sufficient pressure is placed on the sensor pad, such as when a patient lays on the mattress. A heavy/rigid sponge or plastic layer 220 separates the bottom layer 212 from the contact plates 213 and 215.

The under-mattress patient monitoring sensor pad configurations shown in FIG. 2A and FIG. 3 accommodate the environment beneath a mattress or cushion. In a non-limiting example, the patient sits or lays six inches (15 cm) or more above the under-mattress patient monitoring sensor pad 100, and the mattress or cushion distributes the patient's weight in a way that is not accomplished by conventional sensor pads. As mentioned above, to prevent false alarms or a non-functioning sensor pad, the bottom portion of the under-mattress patient monitoring sensor pad 100 is made with rigid layers beneath the detection layers (e.g. the detection layers 113, 114, 115 in FIG. 2A and 213, 214, 215 in FIG. 3). The rigid layers (such as the bottom-most layers 125, 220, 212 and/or 112) make a level or flat surface below the detection layers, no matter what the type of support is under the mattress or cushion.

The configurations for under-mattress patient monitoring sensor pads described above leave the top portion of the sensor pads soft, to be able to react to changes in the weight above the sensor pad. The flexibility in the detection layers (e.g. layers 113, 114, 115 in FIG. 2A and 213, 214, 215 in FIG. 3) allows the contact plates 113 and 115 to contact one another when a patient lays or sits on the mattress or cushion above the sensor pad.

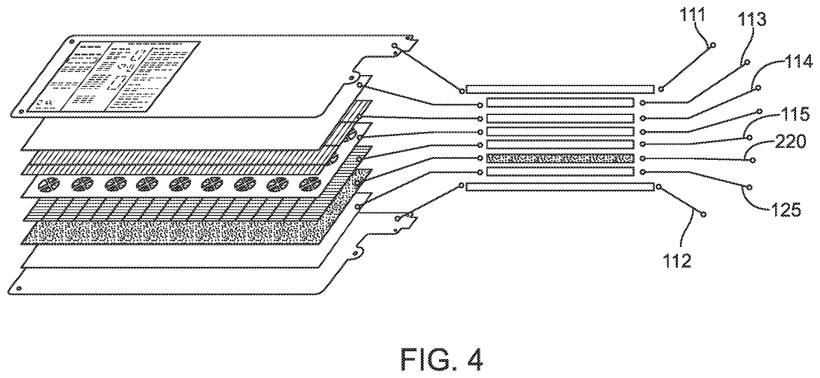

FIG. 4 shows another embodiment of an under-mattress pad 300 that includes an additional hard layer of material 305 between the upper PVC layer 311 and the contact plate 314. The under-mattress pad 300 includes the upper PVC layer 311, the hard layer of material 305, a top contact plate 313, a bottom contact plate 315, a rigid layer 320, a heavy/rigid sponge or plastic layer 320, a bottom plastic layer 325, and a bottom PVC layer 312.

An under-mattress patient monitoring sensor pad can include circuitry to detect contact between the sensor plates 113 and 115. Also, circuitry can be included in a under-mattress patient monitoring sensor pad that signals a nurse call system once the absence of contact between the sensor plates is detected, and the patient is standing up or otherwise not laying or sitting on the mattress over the under-mattress patient monitoring sensor pad.

When using an under-mattress patient monitoring sensor pad, as described herein, the sensor pad may sit beneath the patient's hips under the mattress. Detection of the patient is best when the under-mattress patient monitoring sensor pad sits under the patient's hip. However, because patients vary in size and can move about in bed, an under-mattress patient monitoring sensor pad can be large enough to accommodate a wide range of patients. The fact that an under-mattress patient monitoring sensor pad is not subject to questionable hygiene situations as often and can be made of more robust materials allows for an under-mattress patient monitoring sensor pad as described above to be used for multiple patients, as well as for longer periods of time, for example multiple months or a year.

It should be appreciated that the sensor pad is not limited to use with a bed or bed mattress. The sensor pad can be used in combination with any item that a patient lays on or sits in, including a bed, chair, couch, and the like. In this regards, the size and shape of the sensor pad can vary to suit the item with which it is used.

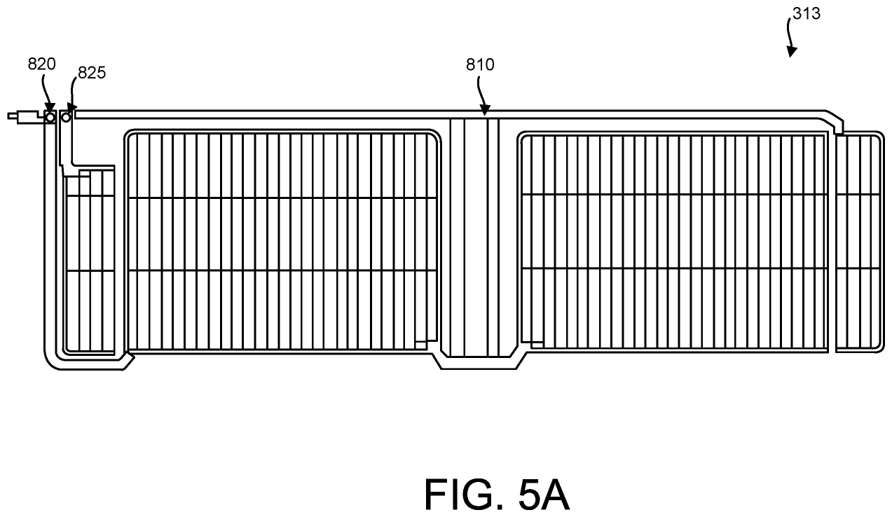

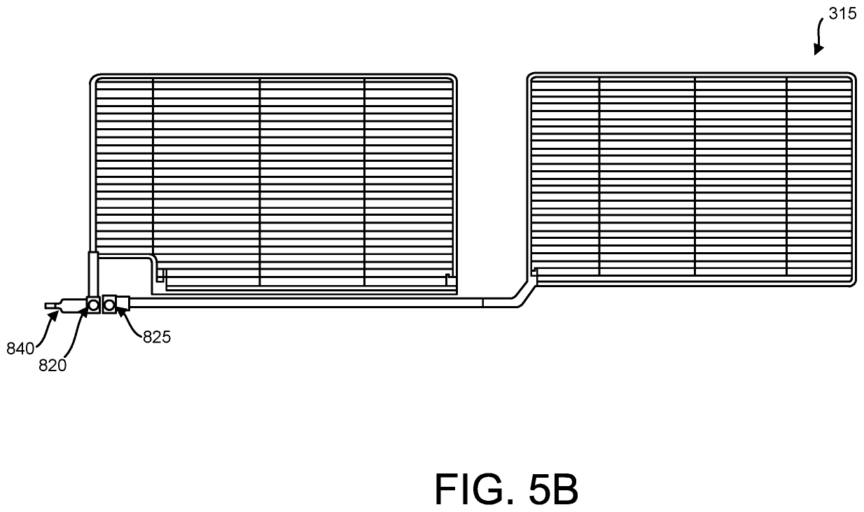

FIG. 5A shows another embodiment of a top contact plate 313 which may be part of an over-mattress or an under-mattress patient monitoring sensor pad. The top contact plate 313 may comprise a metallic element and an organic element, for example silver and carbon, to reduce the resistance of a current through the top contact plate 313. The top contact plate 313 can be separated into two half-pad zones, left and right. The top contact plate 313 may include an insulator 810 in electrical connection with the two zones, to prevent short circuits. Additionally, the top contact plate 313 may include an outer edge zone 820 and an inner edge zone 825. The outer edge zone 820 and the inner edge zone 825 can be configured to detect when a patient is sitting on the edge of the bed, as well as whether the patient has left one half of the bed. In some embodiments, an early alarm or pre-alert feature may be triggered if the outer edge zone 820 and/or the inner edge zone 825 are triggered for at least a few seconds and half of the bed is unoccupied. Additionally and/or alternatively, a low false alarm feature may be triggered only when the patient has left the bed, but includes a delay which decreases to zero if the outer edge zones 820 and/or the inner edge zone 825 are triggered.

FIG. 5B shows an embodiment of a bottom contact plate 315, which can be used with the top contact plate 313 of FIG. 5A. The bottom contact plate 315 can also be separated into two half-pad zones, left and right. Additionally, the bottom contact plate 315 may include an outer edge zone 830 and an inner edge zone 835. The outer edge zone 830 and the inner edge zone 835 can be configured to function in the same way as the outer edge zone 820 and the inner edge zone 825 of the top contact plate 313 (i.e. trigger an early alarm and/or a low false alarm feature). The bottom contact plate 315 can also include an on-pad circuit 840.

The on-pad circuit 840 may be configured to provide resistance information and/or contact information regarding the position of the patient, as described above. Alternatively, the on-pad circuit 840 may be configured to control the alert functions directly from the patient monitoring sensor pad. For example, the patient monitoring sensor pad may include a chip (such as a microchip, control chip, etc.) and, in some embodiments, a small battery, such that the monitoring and alarm functions are performed on the patient monitoring sensor pad itself. This allows the patient monitoring sensor pad to function with any existing product on the market. In some embodiments, the on-pad circuit 840 may include, or may be in electrical connection with, a control feature (for example, a button, slide, switch, knob, etc.) configured to allow a user to switch the patient monitoring sensor pad between an early alarm or pre-alert alarm mode and a low false alarm mode, as described above. Additionally and/or alternatively, the patient monitoring sensor pad may include additional zones to detect lack of movement or hyper movement by the patient.

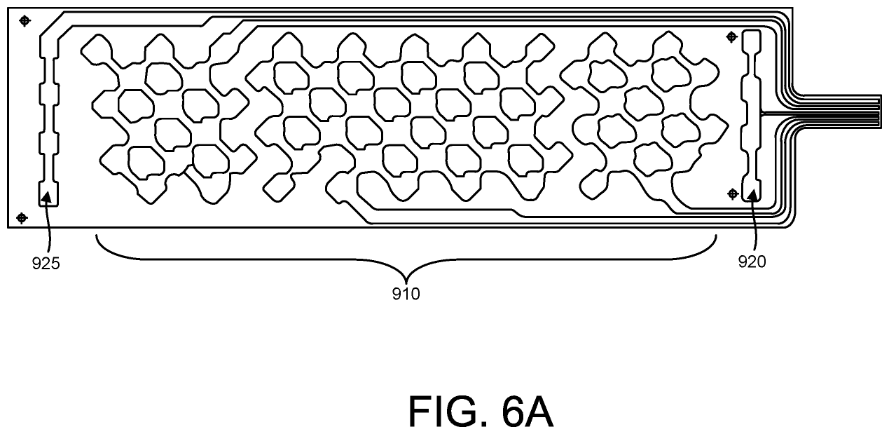

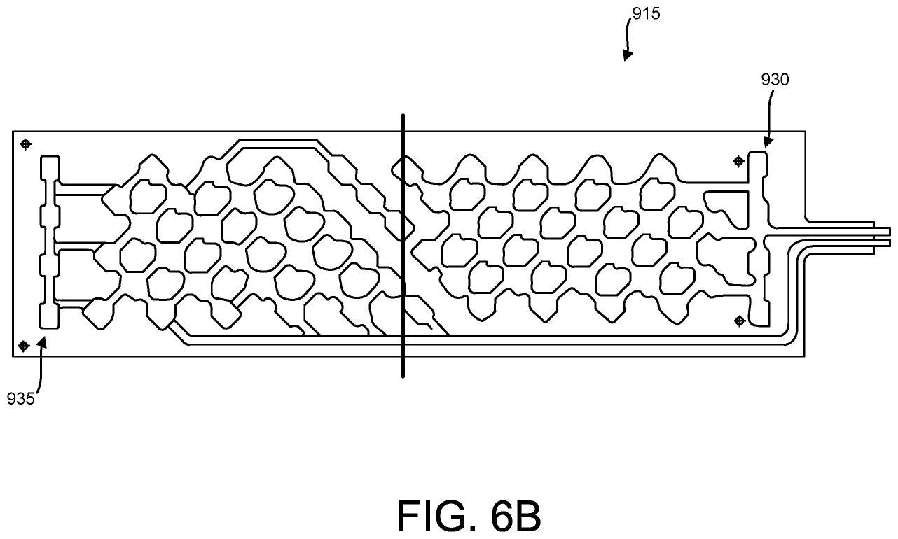

FIG. 6A shows an embodiment of an upper contact plate with movement zones 910, which may be part of an over-mattress or an under-mattress patient monitoring sensor pad. The upper contact plate with movement zones 910 can also include a proximal end zone 920 and a distal end zone 925. The upper contact plate with movement zones 910 can be configured to trigger a warning if lack of movement or hyper movement is detected.

FIG. 6B shows a bottom contact plate with movement zones 915, which can be used with the upper contact plate with movement zones 910 of FIG. 6A. The bottom contact plate with movement zones 915 can also include a proximal end zone 930 and a distal end zone 935. Shown in FIG. 6B is a center point of the bottom contact plate with movement zones 915. The bottom contact plate with movement zones can be visualized as two half-zones on either side of the center point.

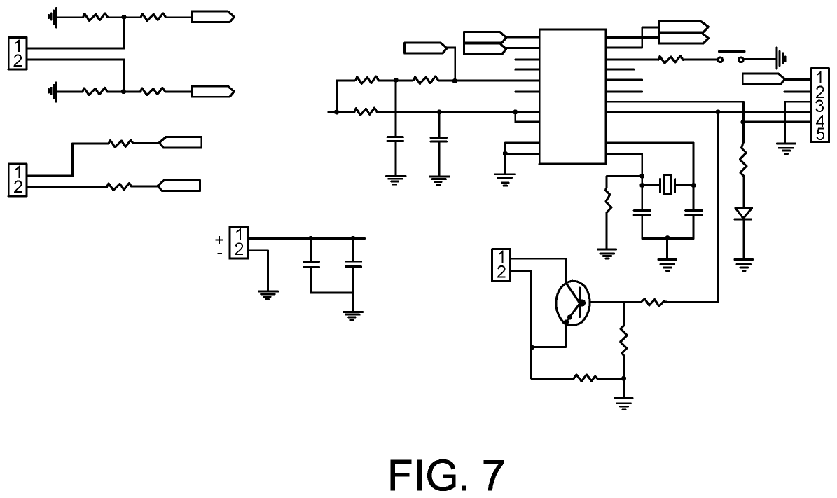

FIG. 7 shows an embodiment of electrical circuitry which can be part of a microchip or control chip included in a patient monitoring sensor pad.

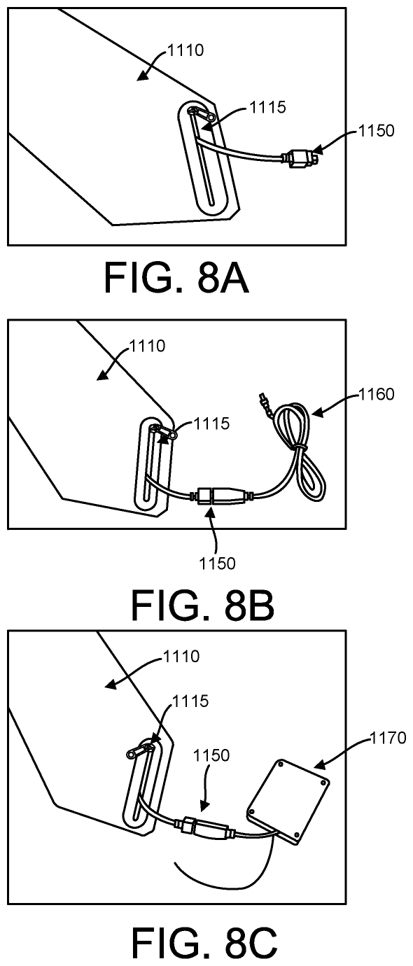

FIGS. 8A-8C show an embodiment of a neck portion 1110 of a patient monitoring sensor pad. As shown in FIG. 8A, the neck portion 1110 can include an aperture 1115 which provides access to an internal cavity. The aperture 1115 is configured to be resealable via a fastening member (not shown). The neck portion 1110 may be configured to contain an electrical contact 1150. The electrical contact 1150 can be positioned on a wire or cord, as shown in FIG. 8A, or may be directly attached to the internal cavity of the neck portion 1110. The electrical contact 1150 may be positioned at the distal end of a cable, such as a break cable. For example, the break cable may be configured to release the electrical contact 1150 from the monitoring device it is connected to if a threshold amount of force is exceeded. In some embodiments, the break cable may be configured to release the electrical contact when approx. 4-8 pounds of force are applied, to prevent damage to the electrical contact 1150. The electrical contact 1150 can be configured to functionally couple with a variety of commercially available products.

As shown in FIG. 8B, the electrical contact 1150 can be connected to (i.e. in electrical connection with) a cord 1160. The cord 1160 may be an electrical contact of a monitoring system or other commercially available product. In some embodiments, the cord 1160 may be an interchangeable cable configured to couple to the electrical contact 1150 at an end proximal to the electrical contact 1150 and to couple to an electrical contact of a monitoring system at an end distal to the electrical contact 1150. For example, the interchangeable cable may include, at the end distal to the electrical contact 1150, at least one of a variety of electrical connectors (e.g. USB, telephone plug, auxiliary cable, etc.) such that the interchangeable cable can allow electrical connection between the patient monitoring sensor pad and a variety of external systems, regardless of the electrical connector required by the external system.

As shown in FIG. 8C, the electrical contact 1150 can be connected to (i.e. in electrical connection with) a wireless unit 1170. The wireless unit 1170 may be a wireless transmitter, wireless receiver, wireless transceiver, wireless transponder, or the like.

The inclusion of a selection of cables or cords and/or a wireless unit compatible with a variety of monitoring systems allows the patient monitoring sensor pad to be used with any monitoring systems, and the cables can be easily fitted at any time if a different monitoring system is required. For example, a patient may be moved between locations and/or may require different monitoring systems at different points in their care. The compatibility of the patient monitoring sensor pad with a variety of monitoring systems saves time and reduces the opportunity for errors or inconsistent communication between monitoring systems, which can be vital in certain patient scenarios.

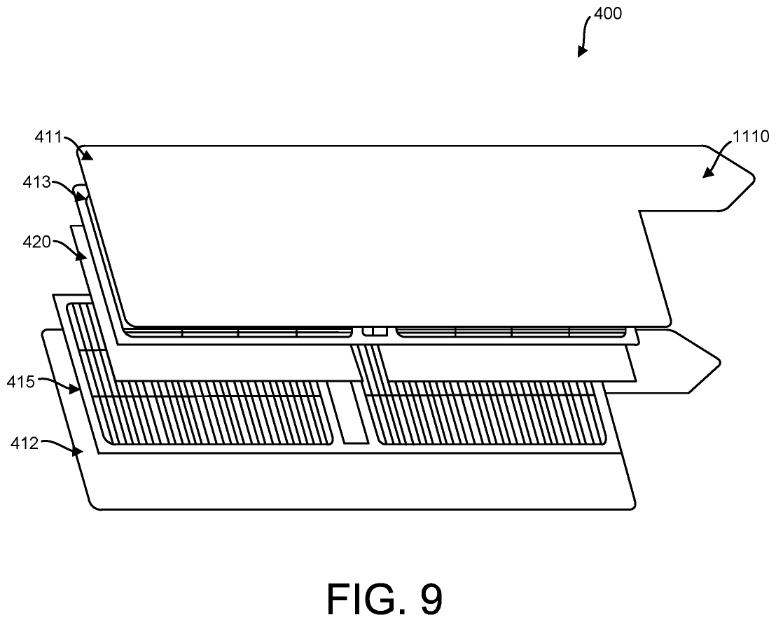

FIG. 9 shows an embodiment of a weight sensing under-mattress pad 400. The weight sensing under-mattress pad 400 includes a top-most PVC layer 411, a top conductive plate 413, a layer of conductive foam 420 (which does not have any holes therethough in an embodiment), a bottom conductive plate 415, a bottom PVC layer 412, and a neck portion 1110. The weight sensing under-mattress pad 400 may include the functions and features of the patient monitoring sensor pads and embodiments thereof described above. A key difference between under-mattress pads and over-mattress pads is that the weight sensing under-mattress pad 400 must be configured to discount the weight of the mattress, and therefore to detect a change in weight rather than a simple on/off function or contact detection. The weight sensing under-mattress pad 400 must be configured to calibrate in order to measure three states: 1) pad empty (no weight); 2) pad with mattress only (bed empty); and 3) pad with patient and mattress (bed occupied). It is important that the weight sensing under-mattress pad 400 be able to calibrate the ‘bed empty’ setting to the weight level of that with mattress only, and be able to calibrate the ‘bed occupied’ setting to the weight of the mattress and patient, in order to detect changes in weight that would indicate a change of state (for example, bed occupied to bed empty). Additionally, the weight sensing under-mattress pad 400 must be able to detect these changes in different zones, in order to be compatible with the pre-alert alarm feature described above. To achieve this, a layer of conductive foam 420 can be positioned between the top conductive plate 413 and the bottom conductive plate 415.

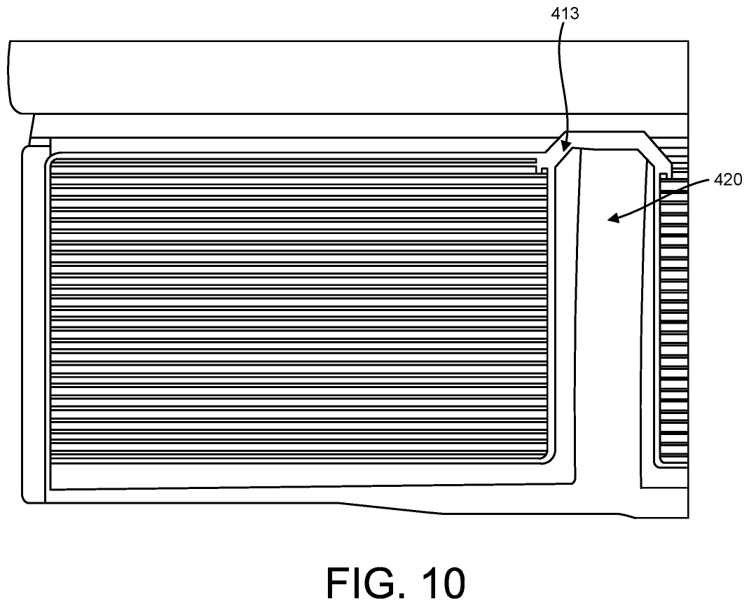

FIG. 10 shows an embodiment of the top conductive plate 413 positioned atop the layer of conductive foam 420. The layer of conductive foam 420 exhibits reduced resistance when pressure is applied. For example, the higher the pressure applied to the layer of conductive foam 420, the lower the resistance will be. Therefore, the weight sensing under-mattress pad 400 must detect a change of resistance in each zone to detect whether the patient is in the center of the bed, on the edge of the bed, or has left the bed completely.

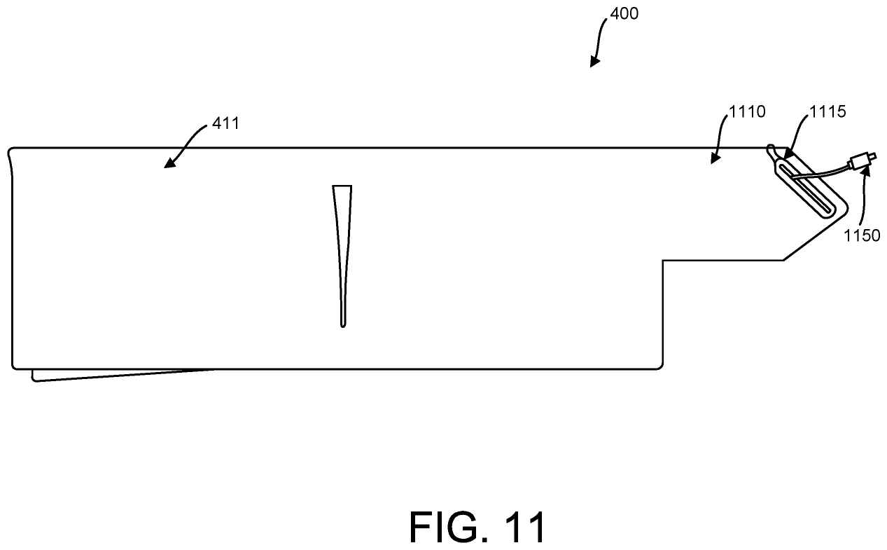

FIG. 11 shows an embodiment of the weight sensing under-mattress pad 400 as assembled. The top-most PVC layer 411 is positioned atop all of the other layers and components, and the bottom PVC layer 412 is positioned below all of the other layers and components. The neck portion 1110 is also shown in FIG. 11, including the aperture 1115 and electrical contact 1150.

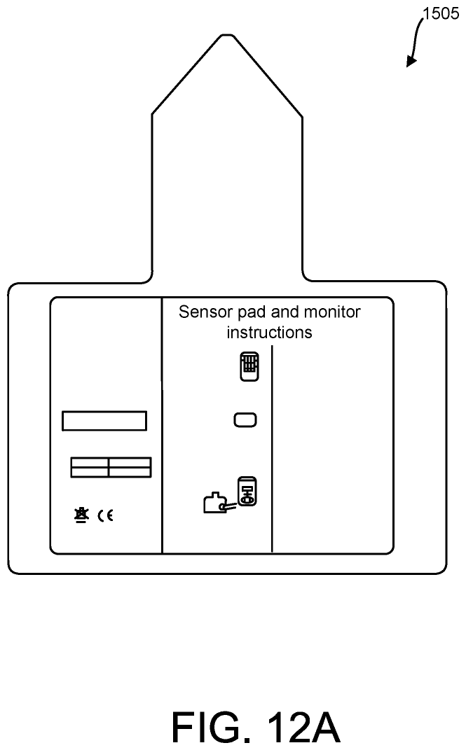

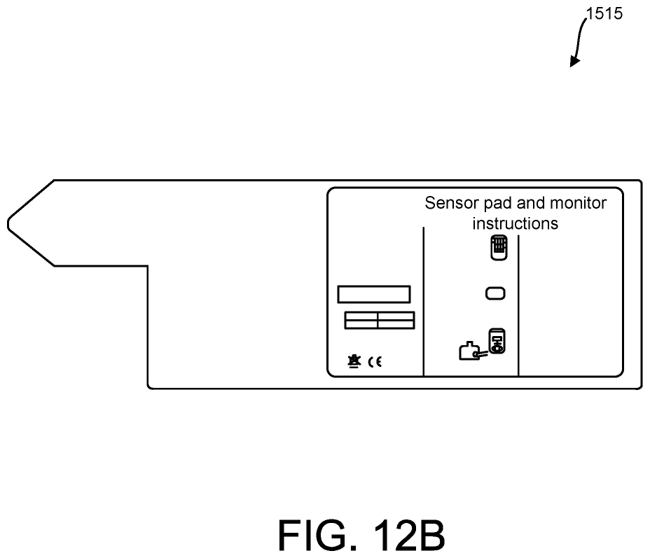

FIG. 12A shows an embodiment of an assembled patient monitoring chair sensor pad 1505, configured for use in a chair or seat. FIG. 12B shows an embodiment of an assembled patient monitoring bed sensor pad 1515, configured for use in a bed or the like.

In any of the embodiments described herein, the conductive foam provides one or more of the following qualities:

The conductive foam 420 is configured to conduct electricity. The conductive foam 420 has an electrical resistance that varies as a function of an amount of force applied to the conductive foam 420. For example, the electrical resistance is higher (such as an almost open circuit) in a situation where no force or less force is applied. The electrical resistance of the conductive foam 420 decreases or lowers as a level of force on the conductive foam 420 increases. That is, the electrical resistance of the conductive foam 420 decreases with increased force or pressure and increases with less force or pressure on the conductive foam 420. The resistance can be a certain level at a default state such as when no force is applied (such as when a person is not present on the mattress or conductive foam 420.) The electrical resistance of the electrically conductive foam decreases as an amount of force on the foam increases and increases as an amount of force on the foam decreases (or vice versa in other embodiments.)

In an embodiment, when power is applied to the system, (such as via a power switch) the system emits an audio signal such as by beeping slowly. A layer of conductive foam 420 (or a pad containing the conductive foam 420) can then be positioned under (or above) the mattress to cause the system to calibrate using a baseline resistance threshold of the conductive foam 420. The sign (such as audio signal) gradually gets faster to indicate the system is calibrating. The system then calibrates to the mattress such as in an empty bed status.

If a weight (such as a person) gets positioned on the bed or mattress, the weight on the conductive foam increases and this is detected by the pad as a lower resistance above a certain threshold (such as a default or baseline resistance threshold), which indicates the bed is occupied. The system may also include an edge area which also can detect if the person is sitting on the edge and about to leave such as via a resistance change. When the person leaves the bed the resistance returns to a calibrated, default level which indicates that just the mattress weight (without the extra weight of the person) is applied to the conductive foam. This indicates an empty bed, which may cause the system to emit alarm, such as an audio or visual alarm. While the bed is in an empty state, the system recalibrates and will keep recalibrating every few minutes.

The disclosed system allows the usage of almost any type of mattress due the fact it can calibrate and discount the weight of the mattress and detect a change of resistance to equal a change in weight (such as the weight of a person on the bed mattress.)

Although described in the context of use with a nursing station, it should be appreciated that the sensor pad and embodiments thereof can be used in any situation where monitoring of a patient or individual is desired. For example, the sensor pad can be communicatively coupled to a Personal Emergency Alarm System (PERS) for on-site assisted living accommodation to a nurses station or off-site alarm relay via the telephone network or internet to family, friends, caregivers, control centers, or any combination thereof. The sensor pads can also be communicatively coupled with nurse call systems in hospitals, nursing homes, and other assisted living facilities.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, methods of use, embodiments, and combinations thereof are also possible. Therefore the spirit and scope of the appended claims should not be limited to the description of the embodiments contained herein.

What is claimed is:

1. A patient monitoring sensor pad, comprising:
a flexible first plate with conductive material printed thereon;
a flexible second plate with conductive material printed thereon, wherein the first flexible plate and second flexible plate comprise zones of conductivity;
a foam layer, wherein the foam layer is an electrically conductive foam and wherein an electrical resistance of the electrically-conductive foam varies as a function of an amount of force applied to the electrically conductive foam;
wherein the zones of conductivity of the first plate are configured to make physical and electrical contact with the zones of conductivity of the second plate when a patient sits or lies on the patient monitoring sensor pad;
a bottom layer below the second plate, the bottom layer being more rigid than the first and second; and
a neck portion extending from the bottom layer, the neck portion comprising an internal cavity, wherein the internal cavity comprises at least one electrical contact and an aperture, the aperture configured to be opened and closed via a fastening member.

2. The patient monitoring sensor pad of claim 1, wherein the bottom layer is Polyvinyl chloride.

3. The patient monitoring sensor pad of claim 1, further comprising a plastic layer between the bottom layer and the second plate.

4. The patient monitoring sensor pad of claim 1, further comprising a top-most layer above the first layer.

5. The patient monitoring sensor pad of claim 1, wherein the top-most layer is Polyvinyl chloride.

6. The patient monitoring sensor pad of claim 1, wherein the neck portion is configured to extend from the top-most layer.

7. The patient monitoring sensor pad of claim 1, wherein the internal cavity further comprises a control chip and a battery.

8. The patient monitoring sensor pad of claim 7, wherein the control chip comprises a monitoring unit and an alarm feature.

9. The patient monitoring sensor pad of claim 1, wherein the electrical resistance of the electrically conductive foam decreases as an amount of force on the foam increases and increases as an amount of force on the foam decreases.

10. The patient monitoring sensor pad of claim 8, wherein the information regarding the position of the patient on the patient monitoring sensor pad comprises an indication of hypermovement, an indication of lack of movement, or an indication of proximity to an edge of the patient monitoring sensor pad.

11. The patient monitoring sensor pad of claim 8, wherein the control chip further comprises a control feature.

12. The patient monitoring sensor pad of claim 1, wherein the at least one electrical contact is an elongate wire in electrical connection with the zones of conductivity.

13. The patient monitoring sensor pad of claim 8, wherein the elongate wire is configured to be functionally coupled to any wired or wireless monitoring unit.

14. The patient monitoring sensor pad of claim 1, further comprising a bed frame, the bed frame being formed of a pair of side rails and a plurality of struts connecting the side rails.

15. The patient monitoring sensor pad of claim 14, further comprising a mattress positioned atop the bed frame such that the pad is positioned between the mattress and the bed frame.

16. A method of using a patient monitoring sensor pad, comprising:
positioning a bed pad atop a bed frame, the bed pad comprising:
(a) a flexible first plate with conductive material printed thereon; and
(b) a flexible second plate with conductive material printed thereon, wherein the first flexible plate and second flexible plate comprise zones of conductivity;
a foam layer, wherein the foam layer is an electrically conductive foam and wherein an electrical resistance of the electrically-conductive foam varies as a function of an amount of force applied to the electrically conductive foam;
wherein the zone of conductivity of the first plate is configured to make physical and electrical contact with the zone of conductivity of the second plate when a patient sits or lies on the patient monitoring sensor pad; and
(c) a neck portion extending from the bottom layer, the neck portion comprising an internal cavity, wherein the internal cavity comprises at least one electrical contact and an aperture, the aperture configured to be opened and closed via a fastening member; and
coupling the bed pad to a monitoring system via the electrical contact within the internal cavity of the neck portion.

17. The method of claim 16, further comprising positioning a mattress atop the bed frame such that the pad is positioned between the mattress and the bed frame.

18. The method of claim 16, further comprising positioning a mattress between the bed frame and the bed pad such that the pad is positioned atop the mattress.

19. The method of claim 16, wherein the electrical resistance of the electrically conductive foam decreases as an amount of force on the foam increases and increases as an amount of force on the foam decreases.
